(19) United States
(12) Patent Application Publication
(10) Pub. No.: US 2012/0203098 A1
(43) Pub. Date: Aug. 9, 2012

(54) ULTRASOUND POWER SUPPLY FOR AN ULTRASOUND TRANSDUCER

(75) Inventors: Balasundara Iyyavu Raju, Tarrytown, NY (US); Shiwei Zhou, Yorktown Heights, NY (US); Shunmugavelu Sokka, Brighton, MA (US)

(73) Assignee: KONINKLIJKE PHILIPS ELECTRONICS N.V., EINDHOVEN (NL)

(21) Appl. No.: 13/501,571

(22) PCT Filed: Sep. 24, 2010
(86) PCT No.: PCT/IB2010/054318

§ 371 Date: Apr. 12, 2012

Related U.S. Application Data
(60) Provisional application No. 61/251,774, filed on Oct. 15, 2009.

(51) Int. Cl. A61N 7/02; H05B 1/02; A61B 5/055

(52) U.S. Cl. 600/411; 601/3; 219/494

(57) ABSTRACT

An ultrasound power supply (100) adapted for supplying electrical power for driving an ultrasound transducer (202, 702) in contact with a subject (208), wherein the ultrasound power supply comprises:—a communications interface (102) adapted for receiving a first temperature measurement of a first volume (211, 318) of the subject and a second temperature measurement of a second volume (214, 320) of the subject;—a controller (108) adapted for modulating the output of electrical power for driving the ultrasound transducer such that via ultrasonic heating by the ultrasound transducer: a. the first temperature measurement is maintained above a first predetermined threshold, b. the first temperature measurement is maintained below a second predetermined threshold, and c. the second temperature measurement is maintained below a third predetermined threshold; and wherein the first predetermined threshold is above the third predetermined threshold.

The invention relates to the control of ultrasound systems, in particular the modulation of electrical power for driving an ultrasound transducer.

Advances in cancer treatment include localized chemotherapy that can reduce systemic side effects to the patient compared to free drug administration. One such method is liposomal encapsulation of cytotoxic drugs such as doxorubicin. Recent research has led to the development of liposomes that are targeted to pathologies and are temperature sensitive. Temperature sensitive liposomes (TSL) can be traditional that are triggered in the range 42° C. to 45° C., or low temperature sensitive liposomes (LTSL) that release their payload in the range 39.5 to 42° C. A variety of methods are available to provide temperature increase to activate the TSLs. These include radio frequency, microwave and ultrasound.

Ultrasound is quickly becoming a desired approach for specific therapeutic interventions. In particular, the use of high intensity focused ultrasound is currently being used as an approach for thermal therapeutic intervention for uterine fibroids and has been examined for possible uses in the treatment of liver, brain, prostate, and other cancerous lesions. Ultrasound therapy for tissue ablation works by insonifying a tissue of interest with high intensity ultrasound that is absorbed and converted into heat, raising the temperature of the tissues. As the temperature rises above 55° C., coagulative necrosis of the tissues occurs resulting in immediate cell death. The transducers used in therapy can be outside the body or be inserted into the body e.g. through blood vessels, urethra, rectum etc. The same transducer could be used for producing non-ablative temperature rises of only a few degrees through suitable adjustment of the power and duration of the sonication, which enables delivery of drugs using TSLs.

U.S. Pat. No. 5,323,779 discloses a pulsed heat-producing device that selectively heats a region in a specific tissue within a patient destroying the tissue. In one embodiment the pulsed heat producing device is a focused ultrasound transducer which concentrates ultrasonic energy at a focal point within the specific tissue.

The invention provides for an ultrasound power supply, an ultrasound system, a computer program product and a method of operating an ultrasound power supply in the independent claims. Embodiments are given in the dependent claims.

Embodiments of this invention may be useful for cancer therapy for prostate cancer treatment using an applicator with an array of ultrasound transducer elements placed in the urethra but is applicable to cancers in other part of the body as well, e.g., breast, liver, brain, and bone. Prostate cancer is frequently found near the periphery of the capsule towards the posterior region. Critical structures such as neurovascular bundles are often located close to the tumor region and need to be preserved.

It is advantageous to activate LTSL in the regions that contain tumors while preserving tissues that are nearby e.g., the nerve bundles in the case of prostate therapy. The therapeutic temperature at the tumor region (39.5° C. to 42° C.) may need to be maintained for a substantial amount of time e.g., 30 minutes, in order for liposomes with a drug payload to be replenished due to circulation. If a continuous sonication scheme is used during this period, it would lead to unwanted therapeutic temperature rises in the critical structures that are located outside and in close proximity to the treatment region. The problem exists for both focused and unfocused transducers wherein critical structures are in close proximity to the treatment spot, e.g., in the case of prostate treatment, the nerve bundles are in close proximity to the tumor.

Previously, treatment of prostate using transurethral ultrasound applicators have been described in prior art wherein the single element transducers are inserted into the urethra to insonify the region in front of it, and sometimes rotated to cover the full cross-section. These transducers are advantageous over focused transducers due to the simplicity and low cost of the design. A transurethral design is advantageous since it provides direct acoustic access to the prostate gland without the effects of intervening tissues. In practice, several transducer elements are placed along the urethral axis in order to cover the 3D volume. The entire treatment process can be performed with the aid of image guidance using Magnetic Resonance Imaging (MRI), ultrasound or other techniques. MRI in particular has the capability to provide information on the temperature rise in the tissues in a spatial region. Such applicators can also be employed to deliver drugs using heat sensitive liposomes.

Embodiments of the invention may enable maintaining the treatment region at temperatures needed for drug delivery through LTSLs while maintaining regions that are outside but in close proximity to temperature levels that are below this threshold. The therapy thus is truly local and may reduce unwanted side effects.

The invention provides for an ultrasound power supply adapted for supplying electrical power for driving an ultrasound transducer in contact with a subject. It is understood that in contact with a subject may mean that the ultrasound transducer is directly in contact with a subject or the ultrasound transducer may be in contact with the subject via an ultrasound conducting medium. An unfocused ultrasound transducer as used herein is defined as an ultrasound transducer which emits ultrasound capable of heating a volume of a subject but is not focused to a specific point. Unfocused ultrasound transducers may have multiple ultrasound transducer elements, but they are not focused. By controlling the phase and amplitude of individual ultrasound transducer elements the volume of ultrasound energy deposited by the unfocused ultrasound transducer may be adjusted to a small degree. The use of multiple ultrasound transducers may also allow the ultrasound to be distributed over a more uniform volume. Unfocused ultrasound transducers may be in the form of a linear array of ultrasound transducer elements, and also may be packaged so that they can be inserted into an orifice of the subject. A focused ultrasound transducer as used herein is an ultrasound transducer which focuses ultrasonic energy to a focal region.

The ultrasound power supply comprises a communications interface adapted for receiving a first temperature measurement of a first volume of the subject and a second temperature measurement of a second volume of the subject. The computer communications interface may be implemented in a variety of ways and may depend upon the method which is used for making the temperature measurement of the first volume and of the second volume. The communications interface may therefore take a variety of forms. For instance the communications interface could be a digital interface, it could be a network connection, it could be an internal bus or interface within a single instrument, or it may even be an analogue interface. For instance thermocouples which supply a voltage or current may be used to send the first temperature measurement and a second temperature measurement to the communications interface. There may be a separate communications interface for receiving the first temperature measurement and a separate communications interface for receiving the second temperature measurement. Alternatively, the communications interface for the first and second temperature measurements may also be combined.

The ultrasound power supply further comprises a controller adapted for modulating the output of electrical power for driving the ultrasound transducer such that the first temperature measurement is maintained, via ultrasonic heating by the ultrasound transducer, above a first predetermined threshold and below a second predetermined threshold. The second temperature measurement is maintained below a third predetermined threshold. The first predetermined threshold is above or equal to the third predetermined threshold. The first volume may be between the ultrasound transducer and the second volume. This embodiment is beneficial, because the first volume can be heated to a temperature which may have a specific effect on the subject. For instance the first predetermined threshold could be a temperature at which drug release occurs. The third predetermined threshold could be a temperature below which there is no effect or lasting effect on the second volume of the subject. This embodiment is beneficial because some temperature activated drugs have a highest chemical reactivity within a predetermined temperature range.

In another embodiment, the ultrasound transducer is an unfocused ultrasound transducer. The first volume is between the ultrasound transducer and the second volume.

In another embodiment, the ultrasound transducer is a focused ultrasound transducer. In another embodiment the controller is adapted for modulating the output of electrical power by gating the output of the electrical power. This modulation of the electrical power may be achieved by modulating the output of electrical power to all of the ultrasound transducer elements which make up an ultrasound transducer. Alternatively the electrical power could be gated for a sub-selection of the ultrasound transducer elements which make up an ultrasound transducer. Another variation of this approach is to vary the duty cycle as a function of time. For instance to increase power, the duty cycle can be increased, and to decrease the power the duty cycle can be reduced.

In another embodiment the controller is adapted for modulating the output of electrical power by continually bearing the power supplied to the ultrasound transducer. This is advantageous, because instead of simply gating the power the power supplied to the ultrasound transducer may be increased or decreased. The electrical power supplied to the ultrasonic transducer may be ramped over time. This embodiment is advantageous because the temperature in the first and second volumes may be more stable than if the power to the ultrasound transducer is simply turned on and off. The amplitude between individual ultrasound transducer elements of an ultrasound transducer may be varied relative to each other to alter the distribution of ultrasound power over time.

In another embodiment the first volume and the second volume are separated by a linear distance of between 0.25 mm and 5 mm. This is advantageous, because the first volume of the subject can be heated above the first predetermined threshold without damaging the second volume of the subject.

In another embodiment, the first temperature measurement is maintained between the first predetermined threshold and second predetermined threshold and the second temperature measurement is maintained below the third predetermined threshold for a period of time between 10 seconds and 1 hour.

In another embodiment, the first predetermined threshold is 39.5 degrees Celsius the second predetermined threshold is 42 degrees Celsius. This embodiment is advantageous, because there are temperature sensitive liposomes that can be used to release a drug payload at these temperatures.

In another embodiment, the first predetermined threshold is 42 degrees Celsius the second predetermined threshold is 45 degrees Celsius. This embodiment is advantageous, because there are temperature sensitive liposomes that can be used to release a drug payload at these temperatures.

In another aspect the invention provides for an ultrasound system. The ultrasound system comprises an ultrasound power supply according to an embodiment of the invention. The ultrasound system further comprises a temperature measurement system adapted for measuring the temperature of the first volume and the second volume. The ultrasound system further comprises an ultrasound transducer. The temperature measurement system may be implemented in a variety of different ways. An invasive technique may be used or a medical imaging method may be used. An example of an invasive technique would be to use thermocouples which are inserted by needles into the first volume and the second volume. The temperature will be measured in the first volume and the second volume using ultrasound techniques. Alternatively, the temperature in the first volume and the second volume may be measured using magnetic resonance thermometry.

In another embodiment the ultrasound system further comprises a magnetic resonance imaging system. The ultrasound power supply is adapted for receiving the first and second temperature measurements from the magnetic resonance imaging system. The magnetic resonance imaging system comprises a magnet adapted for generating a magnetic field for orienting the magnetic spins of nuclei of a subject located within the imaging volume. The imaging comprises the first volume and the second volume. The first volume and the second volume are within the imaging volume. The magnetic resonance imaging system further comprises a radio frequency system comprising a coil adapted for acquiring magnetic resonance imaging data. The coil may be a separate transmission and receive coil or the coil may have an integrated function and be used for both transmission and receiving of signals from the radio frequency system. Magnetic resonance imaging data as used herein is data which is acquired by a magnetic resonance imaging system and which may be used to reconstruct images or other information such as temperature maps that is acquired when a magnetic resonance imaging system is in operation. The magnetic resonance imaging system further comprises a magnetic field gradient coil adapted for spatial encoding of the magnetic spins of nuclei within an imaging volume. The magnetic resonance imaging system further comprises a magnetic field gradient coil power supply adapted for supplying current to the magnetic field gradient coil. The magnetic resonance imaging system further comprises a computer system adapted for constructing images from the magnetic resonance imaging data and for controlling the operation of the magnetic resonance imaging system.

The computer system is adapted for calculating the temperature in the first volume and the second volume using the magnetic resonance imaging data. The computer system may also be used to guide the ultrasound system. The controller of the ultrasound power supply may be the computer system also. The functionality of the computer system is not limited to the magnetic resonance imaging system in some embodiments. A computer system as used herein is a machine adapted for executing machine executable instructions. Examples of a computer system may be a single computer system, an embedded controller, a microcontroller, a network of computers, or a controller. This embodiment is particularly advantageous, because the temperature measurements of the first and second volume are not invasive plus the imaging capability of the magnetic resonance imaging system may be used for guiding the ultrasound system. In another embodiment the temperature measurement system uses thermocouples to measure the temperature of the first volume and the second volume. As was mentioned before the thermocouples may be inserted into the first volume and the second volume of the subject.

In another embodiment the temperature measurement system uses ultrasound to measure the temperature of the first volume and the second volume. This embodiment is advantageous, because it is non-invasive.

In another embodiment the ultrasound system further comprises an injector adapted for injecting a temperature sensitive liposome into the subject. This embodiment is advantageous, because temperature sensitive liposomes can be used to control the delivery of drugs based on the temperature of a region. By controlling the temperature of the first volume, temperature sensitive liposomes can be preferentially delivered to the first volume and not to the second volume.

In another embodiment the ultrasound system further comprises an ultrasound transducer actuator adapted for moving and/or rotating the ultrasound transducer. The ultrasound transducer is adapted for receiving control signals from the ultrasound power supply. The ultrasound power supply is adapted for controlling the temperature of a first volume and a second volume by rotating and/or moving the ultrasound transducer.

The invention provides for a computer program product comprising machine executable instructions for execution by a controller for an ultrasound power supply adapted for supplying electrical power for driving an ultrasound transducer in contact with a subject. The computer program product comprises instructions for performing the step of receiving a first temperature measurement of a first volume of the subject and a second temperature measurement of a second volume of the subject. The computer program product further comprises the step of modulating the output of electrical power, driving the ultrasound transducer such that the first temperature measurement is maintained above a first predetermined threshold and below a second predetermined threshold. The second temperature measurement is maintained below a third predetermined threshold. The first predetermined threshold is above the third predetermined threshold. The first volume may be between the ultrasound transducer and the second volume.

In another embodiment the first temperature measurement and the second temperature measurement are received from a magnetic resonance imaging system. The computer program product may also be executable on a computer system depending upon the embodiment. The computer program product may also be distributed across multiple controllers or computers.

In another aspect the invention provides for a method of operating an ultrasound power supply adapted for supplying electrical power for driving an ultrasound transducer in contact with a subject. The method comprises receiving a first temperature measurement from a first volume of the subject and a second temperature measurement from a second volume of the subject. The method further comprises modulating the output of electrical power driving the ultrasound transducer such that the first temperature measurement is maintained above a first predetermined threshold and below a second predetermined threshold. The second temperature measurement is maintained below a third predetermined threshold. The first predetermined threshold is above or equal to the third predetermined threshold. The first volume may be between the ultrasound transducer and the second volume.

An ultrasound system according to an embodiment of the invention may be used for performing a method for ultrasound mediated drug delivery of a subject. The method comprises the step of accessing treatment planning data of the subject. The treatment planning data is descriptive of the subject's anatomy and may include images such as magnetic resonance images of a treatment zone of the subject. The method further comprises placing an ultrasound transducer adjacent to the treatment zone. The method further comprises monitoring the temperature of a first volume and a second volume. The first and second volumes may be identified in the treatment planning data. The temperature may be monitored in a variety of ways: ultrasound, magnetic resonance imaging, or thermocouples may be used. In the case of magnetic resonance imaging, magnetic resonance imaging thermometry may be used. The first volume may be between the ultrasound transducer and the second volume. The method further comprises injecting temperature sensitive liposomes either intravenously or directly into the treatment zone. The method further comprises maintaining the temperature of the first volume above a first predetermined threshold and below a second predetermined threshold using the ultrasonic transducer. The method further comprises maintaining the temperature of the second volume below a third predetermined threshold. The temperature of the second volume is kept below the third predetermined threshold by controlling the electrical power driving to the ultrasonic transducer. The electrical power driving the ultrasonic transducer may be modulated by gating the power. Alternatively the electrical power to the ultrasonic transducer may be varied continuously to regulate the temperature of the first and second volumes.

In the following preferred embodiments of the invention will be described, by way of example only, and with reference to the drawings in which:

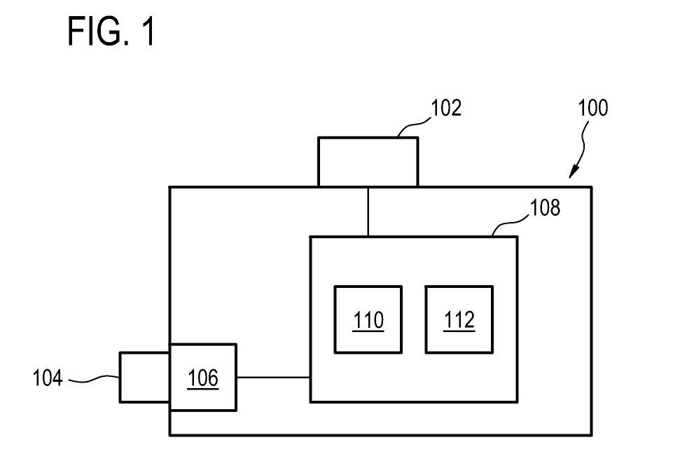

FIG. 1 illustrates an embodiment of an ultrasound power supply;

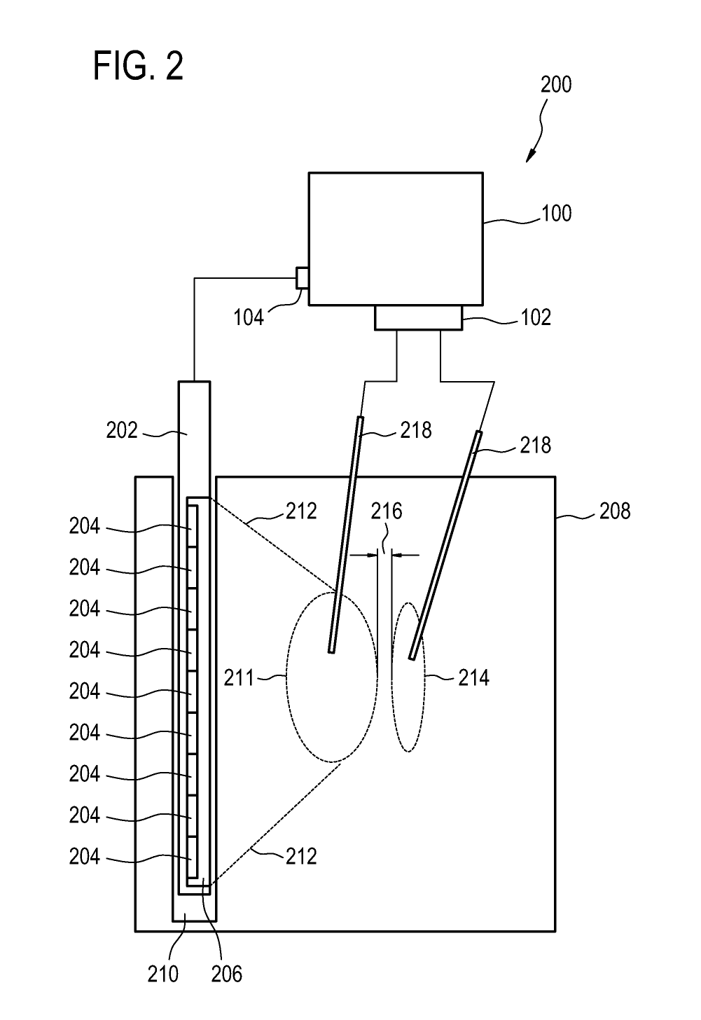

FIG. 2 illustrates an ultrasound system according to an embodiment of the invention;

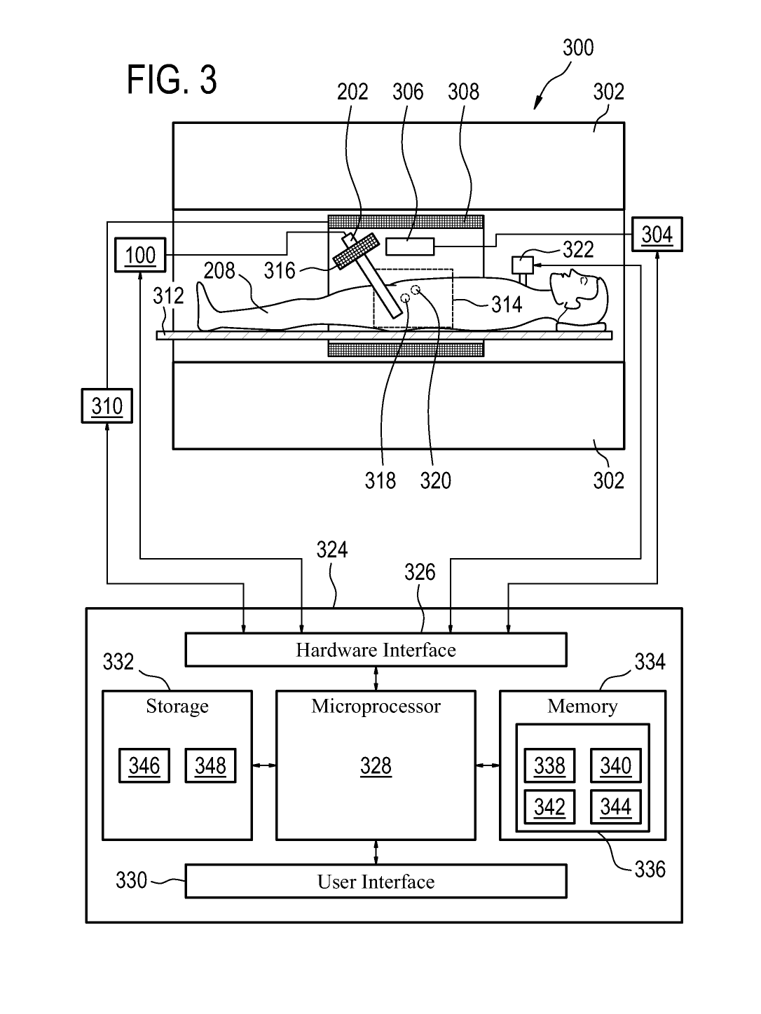

FIG. 3 illustrates an ultrasound system with an integrated magnetic resonance imaging system according to an embodiment of the invention;

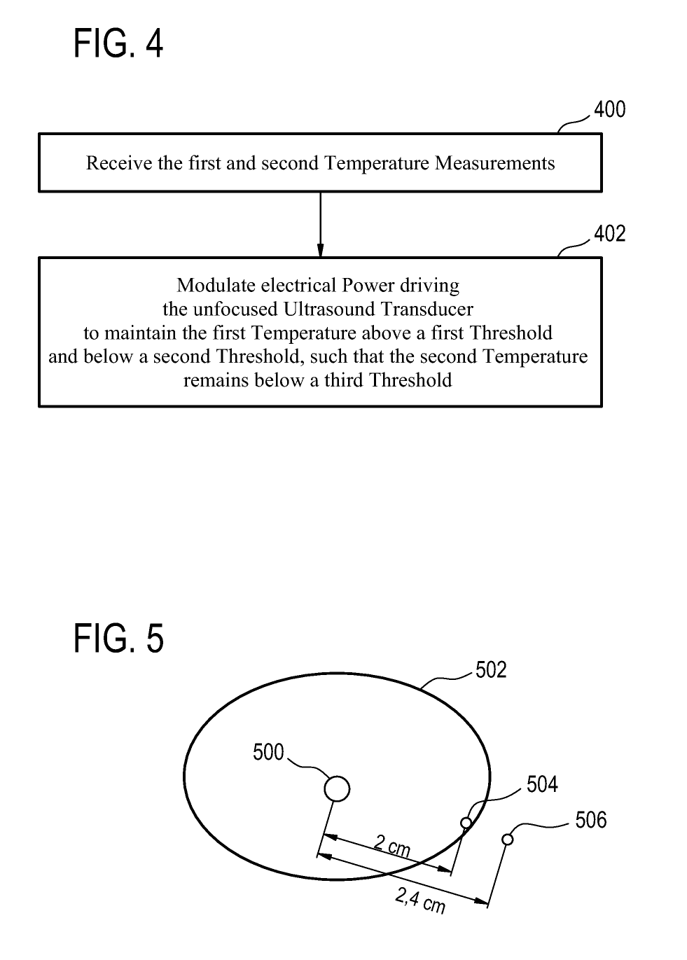

FIG. 4 illustrates an embodiment of a method according to the invention;

FIG. 5 illustrates the geometry of the prostate with the urethra inside;

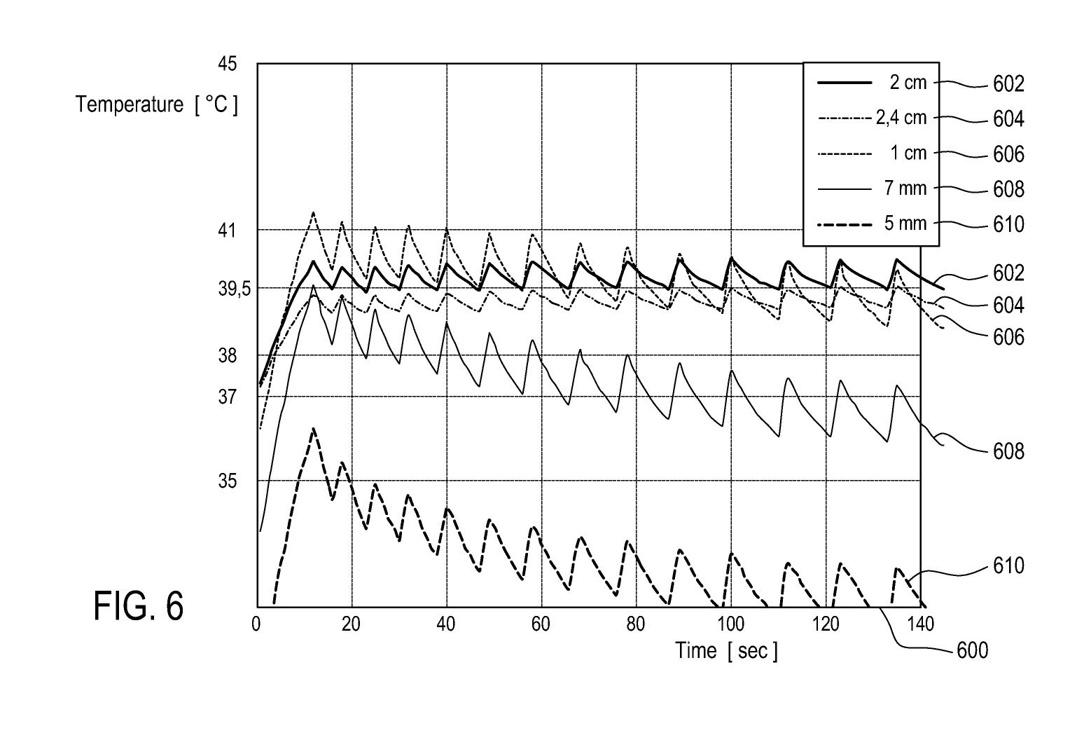

FIG. 6 shows the result of an acoustic and bio heat simulation;

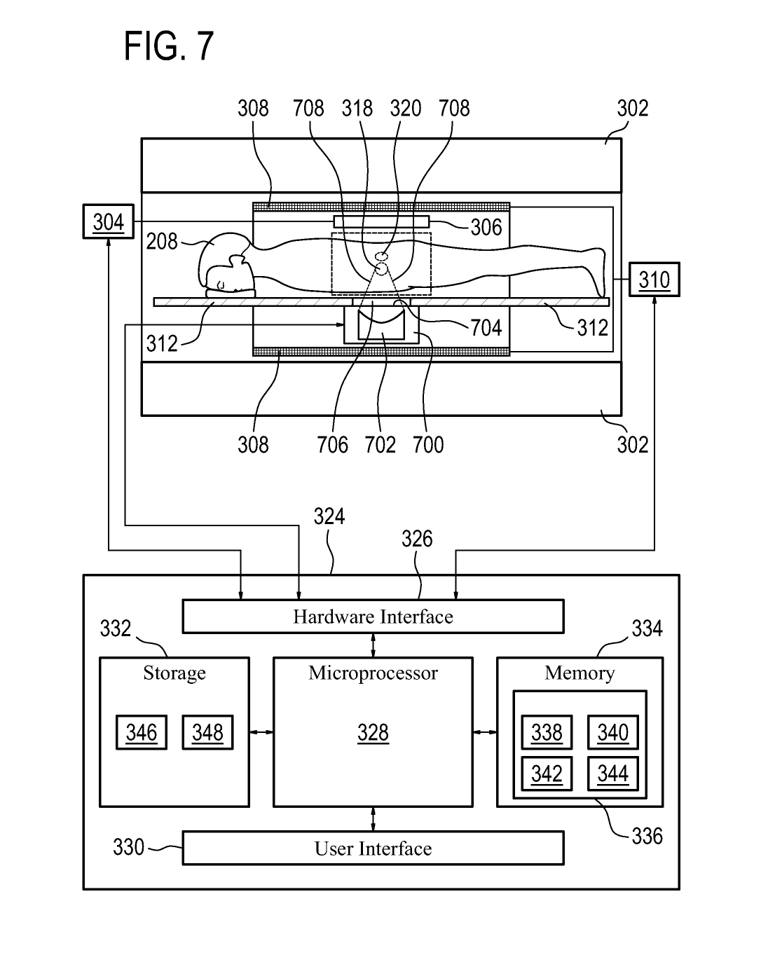

FIG. 7. illustrates an ultrasound system with an integrated magnetic resonance imaging system according to a further embodiment of the invention; and

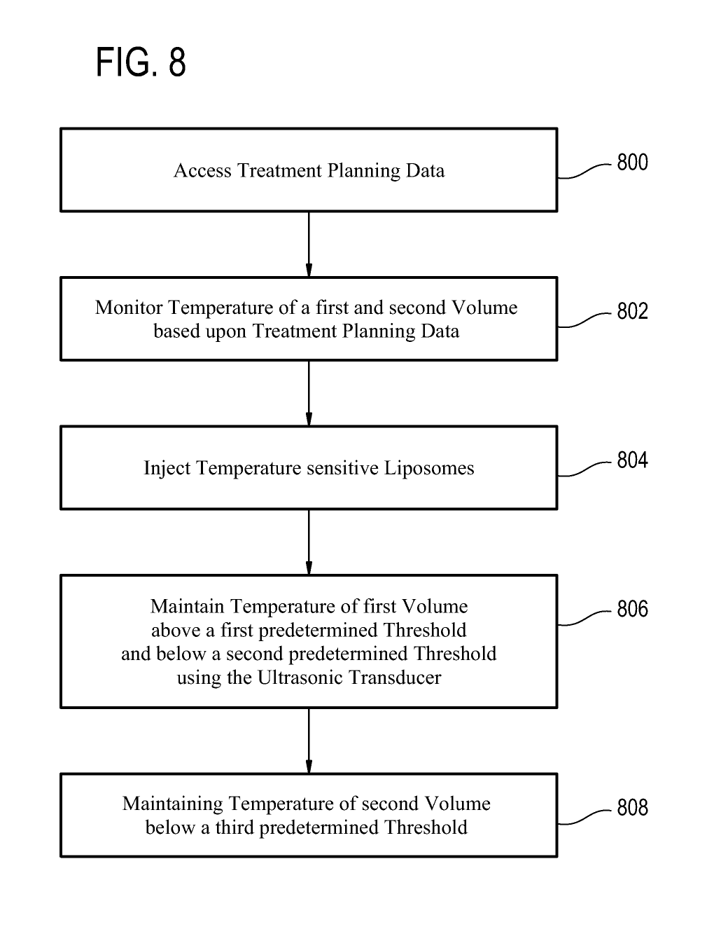

FIG. 8 illustrates a method of ultrasound mediated drug delivery of a subject.

Like numbered elements in these figures are either equivalent elements or perform the same function. Elements which have been discussed previously will not necessarily be discussed in later figures if the function is equivalent.

FIG. 1 shows an embodiment of an ultrasound power supply 100. The ultrasound power supply 100 has a communications interface 102 and an attachment for an ultrasound transducer 104. The attachment for ultrasound transducer 104 is connected to a voltage generator 106. The voltage generator is adapted for producing a voltage which is used for driving ultrasound transducer elements. Also shown in FIG. 1 is a controller 108. The controller 108 receives the first temperature measurement and second temperature measurements from the communications interface 102. The controller 108 is adapted for generating commands for controlling the voltage generator 106. This can be achieved either by digital signals or by generating analogue control signals. The controller 108 comprises a central processing unit 110 which is adapted for executing machine executable instructions. Either within volatile or non-volatile computer memory or on a computer readable medium is contained a computer program product 112. The computer program product 112 contains instructions that the central processing unit 110 uses to generate commands for controlling the voltage generator 106.

FIG. 2 illustrates an ultrasound system according to an embodiment of the invention. Shown in FIG. 2 is an ultrasound power supply 100. The attachment 104 for the unfocused ultrasound transducer is attached to an unfocused ultrasound transducer 202. The unfocused ultrasound transducer 202 illustrated in this fig. is a long cylindrical transducer. There is a cavity 206 filled with an ultrasound medium such as epoxy. Within the cavity 206 is a linear array of ultrasound transducer elements 204. The directionality of the ultrasound produced by the unfocused ultrasound transducer 202 is controlled by moving or rotating the unfocused ultrasound transducer.

The pattern of ultrasound generated by the unfocused ultrasound transducer 202 can be adjusted by controlling the phase and/or amplitude of electrical power applied to each of the ultrasound transducer elements 204. The unfocused ultrasound transducer 202 is inserted into an orifice 210 of a subject 208. Illustrated in this figure is a first volume 211 which is heated by the ultrasound produced by the unfocused ultrasound transducer 202. lines 212 shows the path of ultrasound from the transducer 202 to the first volume 211. Adjacent to the first volume 211 is a second volume 214. The second volume is separated by a distance 216. To measure the temperature within the first volume 211 and the temperature within the second volume 214 thermocouples 218 have been inserted into the subject 208. In this embodiment the communications interface 102 also functions as the electronics for measuring the temperature using the thermocouples 218.

FIG. 2 shows an unfocused ultrasound transducer 202 that can be used in intercavity or interstitial applications. A typical example is prostate therapy where this applicator is inserted through the urethra and placed in the prostatic urethra. The device has 9 elements each having an area of 4 mm×5 mm. When these elements are powered at a suitable frequency, ultrasonic waves are emitted into the tissue. The ultrasound waves get absorbed by the prostate tissues, which cause a rise in the temperature. The temperature rise can be measured by magnetic resonance thermometry techniques. Magnetic resonance thermometry functions by measuring changes in temperature sensitive parameters. Examples of parameters that may be measured during magnetic resonance thermometry are: the proton resonance frequency shift, the diffusion coefficient, or changes in the T1 and/or T2 relaxation time may be used to measure the temperature using magnetic resonance. The proton resonance frequency shift is temperature dependent, because the magnetic field that individual protons, hydrogen atoms, experience depends upon the surrounding molecular structure. An increase in temperature decreases molecular screening due to the temperature affecting the hydrogen bonds. This leads to a temperature dependence of the proton resonant frequency.

FIG. 3 illustrates an ultrasound system with an integrated magnetic resonance imaging system 300 according to an embodiment of the invention. There is a magnet 302 in the bore of the magnet 302 is a subject 208 on a subject support 312. There is a radio frequency coil 306 above the subject 208 which is adapted for acquiring magnetic resonance imaging data within an imaging volume 314. The radio frequency coil 306 is connected to a radio frequency transceiver 304. Also in the bore of the magnet is a magnetic field gradient coil 308 which is connected to a magnetic field gradient power supply 310. An unfocused ultrasound transducer 202 is inserted into an orifice of the subject 208.

There is an ultrasound transducer actuator 316 which is adapted for rotating or moving the unfocused ultrasound transducer 202. Within the imaging volume 314 is the first volume 318 and the second volume 320. The first volume 318 is between the unfocused ultrasound transducer 202 and the second volume 320. There is also an injector 322 connected to the subject 208 which is adapted for injecting a temperature sensitive liposome into the subject. The ultrasound power supply 100 is connected to a hardware interface 326 of a computer system 324. Similarly the magnetic field gradient power supply 310, the injector 322 and the transceiver 304 are also all connected to the hardware interface 326. The computer system 324 also comprises a microprocessor 328 which is connected to a user interface 330, the hardware interface 326, computer storage 332 and computer memory 334. The computer memory 334 contains a computer program product 336.

The computer program product 336 contains modules for operating various functions of the ultrasound system or the magnetic resonance imaging system 300. The computer program product in this embodiment contains a magnetic resonance temperature calculation module 338 which contains machine executable instructions for using magnetic resonance imaging data for calculating temperature maps using magnetic resonance thermometry. The computer program product 336 also comprises code in the form of an ultrasound system control module 340. The ultrasound system control module 340 comprises instructions which allow the microprocessor 328 to send instructions for controlling the ultrasound power supply 100. The computer program product also contains a magnetic resonance system control module 342 which contains instructions which allow the microprocessor 328 for controlling the function and operation of the magnetic resonance imaging system. The computer program product 336 also comprises an image reconstruction module 344. The image reconstruction module 344 contains machine executable instructions which allow the microprocessor 328 to compute images or visualizations of the subject 208 using acquired magnetic resonance imaging data.

The computer storage 332 contains storage for data or for machine executable instructions. For instance the storage may contain an archive of magnetic resonance imaging data 346. The storage 332 may also contain a copy of the computer program product 348.

In operation the apparatus shown in FIG. 3 integrates the functions of the magnetic resonance imaging system 300 and the ultrasound system. An operator may initially take images using the magnetic resonance imaging system and identify features or anatomy of the subject 208 which are intended to be heated with the unfocused ultrasound transducer 202. Next a temperature sensitive liposome 322 may be injected into the subject 208 by the injector 322. After the temperature sensitive liposome has diffused through the body the unfocused ultrasound transducer 202 is used to heat the first region 318 to a temperature which activates the temperature sensitive liposome. The second volume 320 is a sensitive region adjacent to the first volume 318 which could be damaged if the temperature sensitive liposome is activated within the second volume 320. The ultrasonic power supply 100 receives temperature measurements of the first volume 318 and the second volume 320 which were acquired by the magnetic resonance imaging system 300 using magnetic resonance thermometry. The location of the first line 318 may be further controlled by using the ultrasound transducer actuator 316.

FIG. 4 illustrates an embodiment of a method according to the invention. In step 400 the first and second temperature measurements for the first and second volumes respectively are received. In step 402 the electrical power drive in the ultrasound transducer is modulated to maintain the first temperature above a first predetermined threshold and below a second predetermined threshold such that the second temperature remains below a third predetermined threshold. The temperature of the third predetermined threshold temperature is below the first predetermined threshold.

FIG. 5 illustrates the geometry of the prostate with the urethra 500 inside. An unfocused ultrasonic transceiver may be inserted into the urethra 500. Shown is the boundary 502 of the prostate. Point 504 represents a first volume within the boundary of the prostate 502 which would benefit from a heat treatment by an unfocused ultrasonic transceiver. The point labeled 506 represents a second volume which could represent a neurovascular bundle which could be damaged if it were heated above the third predetermined threshold by an unfocused ultrasonic transceiver.

FIG. 5 shows the prostate 502 geometry with the urethra 500 inside. Point 504 may be taken to be a treatment point at the edge of the prostate 502 capsule, 2 cm from the urethra. Point 506 is 2.4 cm away from the urethra and is representative of the neurovascular bundles that are necessary for the patient's potency and is preferably preserved. The aim is to maintain the volume at point 504 for a prolonged period of time at a temperature that causes activation of the LTSLs while simultaneously maintaining the volume at point 506 at a temperature below that threshold. The therapeutic applicator placed in the urethra typically has cooling mechanism in order to preserve the urethra as well as to maintain the transducer temperature to within safe levels.

FIG. 6 shows the result of an acoustic and bio heat simulation. The simulation was chosen to relate the situation illustrated in FIG. 5. The x-axis 600 shows the time in seconds. The y-axis shows the temperature in Celsius of regions that are of varying distance from the urethra. The curved mark 602 is 2 cm from the urethra. This is representative of the first volume 504 shown in FIG. 5. The curve labeled 604 is 2.4 cm away from the urethra and is representative of the second volume 506 shown in FIG. 5. The curve labeled 606 is 1 cm from the urethra, the curve labeled 608 is 7 mm from the urethra and the curve labeled 610 is 5 mm from the urethra. For the simulation used to generate the results shown in FIG. 6, a power modulation was used. The LTSLs were assumed to be injected at time zero. The acoustic simulations were performed using finite element simulations assuming a sonication frequency of 3 MHz with the applicator placed in the urethra. The urethra is cooled with cooling water circulated at a temperature that is maintained at 20° C. The properties of the prostate medium were taken from the literature: density=1050 kg/m3, speed of sound=1530 m/s, ultrasound attenuation=5.3 Np/m/MHz with a linear frequency dependence, specific heat=3639 J/Kg/K, thermal conductivity=0.56 W/m/K, blood perfusion rate=5 Kg/m3/s. The specific heat capacity of blood is taken to be 3650 J/Kg/K. The output acoustic intensity was set to 10 W/cm2. Temperature profiles as a function of time were obtained throughout the region and are plotted for five locations: 5 mm (line 610), 7 mm (line 608), 1 cm (line 606), 2 cm (line 602), and 2.4 cm (line 604) from the urethra in FIG. 5. The control scheme turned the power to the elements on until the point 504 of FIG. 5 at 2 cm reached at least 39.5° C. and continued to hold the power further until the point 506 of FIG. 5 at 2.4 cm just reached 39.5° C. At this time point, the sonication power was turned off and the tissue was allowed to cool. The temperatures at point 504 and point 506 both decreased during this time. Once the temperature at point 504 dropped to 39.5° C., the power was turned on until a point when the temperature at point 506 approached 39.5° C. This process was repeated. As a result the temperature profile at point 504 was maintained to be always above 39.5° C., the temperature for activation of LTSLs, while simultaneously maintaining the temperature at 506 to be below 39.5° C. After about 150 seconds or so, the temperature profiles become predictable and reached a steady state pattern. Hence, the simulations were not continued beyond that time, but the therapy in practice will be continued for a considerably long time such as ten to thirty minutes. In practice this scheme can be achieved using thermometry information from MRI or ultrasound methods.

In the above scheme it can also be seen that locations close to the urethra e.g., at 5 mm (line 610) and 7 mm (line 608) away do not reach therapeutic temperatures and are well below 39.5° C. This is due to the presence of cooling in the urethra. Locations between 7 mm (line 608) and 10 mm (not shown) away can also be preserved by appropriately timing the injection of the drug into the patient, due to the overall decreasing nature of the temperature profile.

As an alternative to modulating the power by switching the power to the unfocused ultrasound transducer on and off, there are several other embodiments:

During the cooling phase the applicator is rotated or moved in order to apply therapy to different locations.

Instead of turning the power ON and OFF, the scheme will gradually increase or decrease the power levels in order to obtain smoother temperature profiles, which reduce the temporal update rate required for thermometry, thereby increasing spatial accuracy of the temperature maps.

FIG. 7 illustrates an ultrasound system with an integrated magnetic resonance imaging system according to a further embodiment of the invention. In this embodiment a high intensity focused ultrasound system 700 is shown. The high intensity focused ultrasound system comprises a focused ultrasound transducer 702. The high intensity focused ultrasound system further comprises an ultrasound window 704 adapted for allowing the passage of ultrasonic waves. Visible in the patient support 312 is an opening 708 which is adapted for allowing the passage of ultrasonic waves. The opening 708 may be adapted for receiving an ultrasonic conducting medium such as gel pads or ultrasonic conducting gel to establish a path from the high intensity focused ultrasound system 700 to the subject 208. The lines 708 mark the path of the ultrasonic waves to the first volume 318. The first volume 318 is between the focused ultrasonic transducer 702 and the second volume 320. With a focused ultrasound transducer 702, the first volume 318 does not need to be between the second volume 320 and the focused ultrasound transducer 702.

FIG. 8 illustrates a method of performing ultrasound mediated drug delivery. An ultrasound system according to an embodiment of the invention may be used for performing this method. In step 800 treatment planning data is accessed. The treatment planning data may contain anatomical data describing the volume or volumes of the subject to be treated. The treatment planning data may also contain data which describes the temperature range to which a first volumes of the subject is heated. Additionally the treatment planning data may also contain data which describes the duration the first volume of the subject is heated to. The treatment planning data may also contain data which describes a second volume of the subject and a maximum temperature to which the second volume may be heated. In step 802 the temperature of a first volume and a second volume is monitored based upon the treatment planning data. The temperature may be monitored continuously or it may be monitored periodically. In step 804, temperature sensitive liposomes are injected into the subject. The temperature sensitive liposomes may be injected intravenously or they may be injected directly into the first volume. Alternatively the temperature sensitive liposomes may be injected into an adjacent volume. The adjacent volume is a volume of the subject that is adjacent to the first volume. In step 806, the temperature of the first volume is maintained between a first predetermined threshold and a second predetermined threshold by heating using the ultrasonic transducer of the ultrasonic system. In step 808, the temperature of the second volume is maintained below a third predetermined threshold. The temperature of the second volume is maintained below the third predetermined threshold by regulating the electrical power delivered to the ultrasonic transducer. The power to the ultrasonic transducer may be gated or it may be varied continuously. For example, the power to the ultrasonic transducer may be a ramp function that varies as a function of time.

1. An ultrasound power supply adapted for supplying electrical power for driving an ultrasound transducer in contact with a subject, wherein the ultrasound power supply comprises:
a communications interface adapted for receiving a first temperature measurement of a first volume of the subject and a second temperature measurement of a second volume of the subject;
a controller adapted for modulating the output of electrical power for driving the ultrasound transducer such that via ultrasonic heating by the ultrasound transducer:
a. the first temperature measurement is maintained above a first predetermined threshold,
b. the first temperature measurement is maintained below a second predetermined threshold, and
c. the second temperature measurement is maintained below a third predetermined threshold; and
wherein the first predetermined threshold is above the third predetermined threshold.

2. The ultrasound power supply of claim 1, wherein the ultrasound transducer is an unfocused ultrasound transducer; and wherein the first volume is between the ultrasound transducer and the second volume.

3. The ultrasound power supply of claim 1, wherein the controller is adapted for modulating the output of electrical power by gating the output of the electrical power to at least one of the elements of the ultrasound transducer

4. The ultrasound power supply of claim 1, wherein the controller is adapted for modulating the output of electrical power by continually varying the power supplied to at least one of the elements of the ultrasound transducer.

5. The ultrasound power supply of claim 1, wherein the first volume and the second volume are separated by a linear distance of between 0.25 mm and 5 mm.

6. The ultrasound power supply of claim 1, wherein the first temperature measurement is maintained between the first predetermined threshold and second predetermined threshold and the second temperature measurement is maintained below the third predetermined threshold for a period of time between 10 seconds and 1 hour.

7. The ultrasound power supply of claim 1, wherein the first predetermined threshold is 39.5 degrees Celsius, and wherein the second predetermined threshold is 42 degrees Celsius.

8. An ultrasound system, the ultrasound system comprising:
an ultrasound power supply according to any one of the preceding claims;
a temperature measurement system adapted for measuring the temperature of the first volume and the second volume; and
an ultrasound transducer.

9. The ultrasound system of claim 8, wherein the ultrasound system further comprises a magnetic resonance imaging system, wherein the ultrasound power supply is adapted for receiving the first and second temperature measurements from the magnetic resonance imaging system, wherein the magnetic resonance imaging system comprises:
a magnet adapted for generating a magnetic field for orientating the magnetic spins of nuclei of a subject located within an imaging volume, wherein the imaging region comprises the first volume and the second volume;
a radio frequency system comprising a coil adapted for acquiring magnetic resonance imaging data;
a magnetic field gradient coil adapted for spatial encoding of the magnetic spins of nuclei within the imaging volume;
a magnetic field gradient coil power supply adapted for supplying current to the magnetic field gradient coil; and
a computer system adapted for constructing images from the magnetic resonance imaging data and for controlling the operation of the magnetic resonance imaging system, wherein the computer system is adapted for calculating the temperature in the first volume and the second volume using the magnetic resonance imaging data.

10. The ultrasound system of claim 8, wherein the temperature measurement system uses thermocouples to measure the temperature of the first volume and the second volume.

11. The ultrasound system of claim 8, wherein the temperature measurement system uses ultrasound to measure the temperature of the first volume and the second volume.

12. The ultrasound system of claim 8, wherein the ultrasound system further comprises an injector adapted for injecting a temperature sensitive liposome into the subject.

13. The ultrasound system of claim 8, wherein the ultrasound system further comprises an ultrasound transducer actuator adapted for moving and/or rotating the ultrasound transducer, wherein the ultrasound transducer is adapted for receiving control signals from the ultrasound power supply, wherein the ultrasound power supply is adapted for controlling the temperature of the first volume and the second volume by rotating and/or moving the ultrasound transducer.

14. A computer program product comprising machine executable instruction for execution by a controller for an ultrasound power supply adapted for supplying electrical power for driving an ultrasound transducer in contact with a subject, wherein the computer program product comprises instructions for performing the steps of:
receiving a first temperature measurement of a first volume of the subject and a second temperature measurement of a second volume of the subject;
modulating the output of electrical power driving the ultrasound transducer such that via ultrasonic heating by the ultrasound transducer:
a. the first temperature measurement is maintained above a first predetermined threshold,
b. the first temperature measurement is maintained below a second predetermined threshold, and
c. the second temperature measurement is maintained below a third predetermined threshold; and
wherein the first predetermined threshold is equal to or above the third predetermined threshold.

15. A method of operating an ultrasound power supply adapted for supplying electrical power for driving an ultrasound transducer in contact with a subject, wherein the method comprises:
receiving a first temperature measurement from a first volume of the subject and a second temperature measurement from a second volume of the subject;
modulating the output of electrical power driving the ultrasound transducer such that via ultrasonic heating by the ultrasound transducer:
a. the first temperature measurement is maintained above a first predetermined threshold,
b. the first temperature measurement is maintained below a second predetermined threshold, and
c. the second temperature measurement is maintained below a third predetermined threshold; and
wherein the first predetermined threshold is equal to or above the third predetermined threshold.
